(19) United States
(12) Patent Application Publication
(10) Pub. No.: US 2025/0134915 A1
(43) Pub. Date: May 1, 2025

(54) METHODS AND SYSTEMS FOR INDUCING APOPTOSIS OF ADIPOSE CELLS

(71) Applicant: Cryosa, Inc., Arden Hills, MN (US)

(72) Inventor: Donald A. Gonzales, Folsom, LA (US)

(21) Appl. No.: 18/919,177

(22) Filed: Oct. 17, 2024

Related U.S. Application Data
(60) Provisional application No. 63/593,937, filed on Oct. 27, 2023.

(51) Int. Cl. A61K 31/7016; A61K 31/4155; A61K 31/437; A61K 31/4412; A61K 31/444; A61K 31/496; A61K 31/5025; A61K 31/517; A61K 31/519; A61K 31/5365; A61K 31/575; A61K 45/06; C12N 15/113

(52) U.S. Cl. CPC A61K 31/7016; A61K 31/4155; A61K 31/437; A61K 31/4412; A61K 31/444; A61K 31/496; A61K 31/5025; A61K 31/517; A61K 31/519; A61K 31/5365; A61K 31/575; A61K 45/06; C12N 15/1138; C12N 2310/11

(57) ABSTRACT

Methods and systems for inducing apoptosis of adipose cells are disclosed herein. In some embodiments, the method comprises identifying a target area comprising the adipose cells and injecting a therapeutic agent at the target area. Injecting the therapeutic agent can inhibit a protein kinase B (Akt) pathway of the adipose cell, induce a proinflammatory response of adipose cells at the target area, and/or initiate lipolysis/cell death of the adipose cells at the target area. In doing so, a volume of the adipose cells at the target area can be reduced.

### CROSS-REFERENCE TO RELATED APPLICATIONS

The present application claims the benefit of U.S. Provisional Patent Application No. 63/593,937, filed Oct. 27, 2023, and titled “METHODS AND SYSTEMS FOR INDUCING APOPTOSIS OF ADIPOSE CELLS,” the disclosure of which is incorporated herein by reference in its entirety.

### TECHNICAL FIELD

This disclosure relates to methods and systems for inducing apoptosis of adipose cells.

### BACKGROUND

Obstructive sleep apnea (OSA) is a condition characterized by repeated episodes of partial or complete obstruction of the upper airway during sleep, leading to disrupted sleep and various health complications. One option to treat such a condition involves cooling the tissue to a range that will damage adipose cells, thereby initiating the migration of macrophages to surround the adipose cell. Once around the cell, these macrophages secrete pro-apoptotic factors that induce the adipose cell to undergo cell death. Methods for inducing adipose cell death via cooling, such as adipose cryolysis, are often used to selectively reduce adipose tissue for cosmetic purposes and to treat OSA by targeting adipose cells in the oropharyngeal area.

Cooling or thermally treating adipose cells, however, has several limitations. These methods are restricted to the superficial layers of fat and cannot be used around vital structures. The procedure requires a significant amount of time and can damage superficial layers, affecting taste, sensory nerves, skin, and muscle. Additionally, thermal treatment can create pain and numbness and may only be performed under general anesthesia. These limitations highlight the need for alternative methods that can effectively induce adipose cell death without the drawbacks associated with these techniques.

### BRIEF DESCRIPTION OF THE DRAWINGS

Features, aspects, and advantages of the presently disclosed technology may be better understood with regard to the following drawings.

FIG. 1 is a flow diagram illustrating a method for inducing apoptosis of adipose cells, in accordance with embodiments of the present technology.

FIG. 2A illustrates a target area of a human patient, in accordance with embodiments of the present technology.

FIG. 2B illustrates a target area of a human patient, in accordance with embodiments of the present technology.

FIG. 3 illustrates a schematic cross-sectional view of an adipose cell and the underlying mechanism that occurs when a therapeutic agent is injected, in accordance with embodiments of the present technology.

FIG. 4 is a flow diagram illustrating a method for inducing apoptosis of adipose cells, in accordance with embodiments of the present technology.

FIG. 5 illustrates a schematic cross-sectional view of an macrophage and the underlying mechanism that occurs when a therapeutic agent is injected, in accordance with embodiments of the present technology.

FIG. 6 illustrates a Crown-like Structure (CLS) around an adipose cell, in accordance with embodiments of the present technology.

FIG. 7 illustrates a schematic cross-sectional view of groups of adipose cells and the underlying mechanisms that occur when therapeutic agents are injected, in accordance with embodiments of the present technology.

FIG. 8 is a flow diagram illustrating a method for inducing apoptosis of adipose cells, in accordance with embodiments of the present technology.

FIG. 9 is a flow diagram illustrating a method for inducing apoptosis of adipose cells, in accordance with embodiments of the present technology.

A person skilled in the relevant art will understand that the features shown in the drawings are for purposes of illustration, and variations, including different and/or additional features and arrangements thereof, are possible.

### DETAILED DESCRIPTION

### I. Overview

The present technology is generally directed to a method of inducing adipose cells (e.g., adipocytes, lipocytes, fat cells, white fat cells, brown fat cells, marrow fat cells, etc.) to preferentially go through cell death, lipolysis, or apoptosis. Current methods used for inducing adipose cell death typically require cooling the target area (and/or the therapeutic agent) to a range that will damage the adipose cell, and therein initiate migration of macrophages to surround the adipose cell. Once around the cell, these macrophages secrete pro-apoptotic factors that will induce the adipose cell to go through cell death. Methods for inducing adipose cell death via cooling (e.g., adipose cryolysis) are often used to selectively reduce adipose tissue for cosmetic purposes and to treat obstructive sleep apnea (OSA) by targeting adipose cells in the oropharyngeal area. Adipose cryolysis (i) is limited to the superficial layers of fat, (ii) cannot be used around vital structures, (iii) requires a large amount of time, (iv) can damage superficial layers to affect taste, sensory nerves, skin muscle, etc., (v) can create pain and numbness, and (vi) may only be performed under general anesthesia.

Embodiments of the present technology address at least some of the above-described issues for inducing cell death of adipose cells that use a cooling technique and initially “injure” adipose cells. As used herein, an “injured cell” can refer to any adipose cell that is thermally treated (e.g., cooled). Embodiments of the present technology include methods and systems for inducing apoptosis of adipose cells chemically, without thermal treatment (e.g., without cooling the target area, cooling the therapeutic agent, etc.). Exemplary methods can comprise identifying a target area including the adipose cells and injecting a therapeutic agent at the target area. Injection of the therapeutic agent(s) allows the agent(s) to act locally and not systemically, reducing the potential of unwanted side effects. For example, a health care practitioner or user can identify the target area and inject the therapeutic agent at the target area while the therapeutic agent and the adipose cells at the target area are at room temperature or a temperature of at least 10° C. As explained herein, the injected therapeutic agent can act similarly to factors released by Adipose Tissue Macrophages (ATMs) that induce adipose cells to undergo lipolysis and/or cell death. These agents can inhibit a protein kinase B (Akt) pathway of the adipose cells, induce M2 macrophage to macrophage conversion, act on macrophages by activating the macrophage-inducible C-type lectin (Mincle) receptor at the target area and/or initiate cell death of the adipose cells, which in turn can reduce a volume of the adipose cells at the target area. Once adipose cell death has occurred, signals will be released by the dead adipose tissue to attract additional M1 macrophages in a feed forward pathway such that additional fat will be reduced. These agents can also inhibit glucocorticoid effects in macrophages by blocking the activity and/or reducing the number of the glucocorticoid receptors (GRs) on the ATM, limiting the inhibition of endogenous or exogenous steroids to induce adipose cell death.

In some embodiments, a desired target volume is established in a treatment plan based on the amount of fat cells in the target area and a desired outcome. As an example, for treating OSA, the desired target volume can be determined by calculating the amount of submucosal fat cells in the target area that need to be removed to permit relief from the OSA symptoms. The desired target volume can also account for fat cells needed in the target area to preserve surrounding tissue. Other examples include removing fat for cosmetic purposes, removing visceral fat, removing breast fat, and removing fat in the upper and lower eyelids. Removing fat in the upper and lower eyelids is particularly challenging due to the vital organs in close proximity (e.g., the eyes, nerves, blood vessels). In some embodiments, a corresponding method comprises injecting a therapeutic agent at the target area, such that the M2 macrophages at the target area are activated by the therapeutic agent and become proinflammatory (M1). The proinflammatory macrophages are induced to surround the adipose cells as a Crown-like Structure (CLS) and can release a pro-apoptotic factor which inhibits the protein kinase B (Akt) pathway of the adipose cells and/or initiates cell death of the adipose cells at the target area.

Embodiments of the present technology bypass initially injuring adipose cells (e.g., via active cooling of target areas, therapeutic agents, etc.) by injecting agents that (i) directly induce the adipose cell to go through apoptosis (Akt-inhibitors), and/or (ii) activate the M2 macrophages to become proinflammatory M1 macrophages, surround the adipose cell, and induce apoptosis of the adipose cell. When an adipose cell is dead or dying, the cell typically attracts M1 macrophages to the surrounding area CLS and causes M2 macrophages in the surrounding area to convert to M1 macrophages. As explained herein, the M2 macrophages become proinflammatory M1 macrophages and surround the non-injured adipose cell in a CLS. M1 macrophages in the CLS can release factors that block the protein kinase B (Akt) pathway, which can act directly on the adipose cells and cause the adipose cells to become insulin resistant and/or promote apoptosis and/or cell death.

In some embodiments, an agent is locally injected that acts on the Mincle receptor as a ligand (e.g., trehalose-6,6′-dimycolate (TDM), lipopolysaccharide (LPS), etc.) to induce macrophages to release factors that initiate cell death of adipose tissue. Additionally or alternatively, the therapeutic agent injected can induce the expression of immune receptors on macrophages and/or shift M2 macrophages to macrophages to undergo a proinflammatory response. For example, the injection of palmitate or palmitic acid can promote the expression of the Mincle receptor, which can, in turn, lead to a generally higher proliferation of proinflammatory macrophages at the target area. The injection of LPS can promote the shift of M2 macrophages to macrophages, inducing a proinflammatory response. The proinflammatory response can promote formation of the CLS that induces adipose cell death. In other embodiments, factors are injected that locally block glucocorticoid activity, such as an antagonist to the GR, or that otherwise decrease production of the GRs in macrophages. For example, a GR antagonist can include mifepristone, metyrapone, ketoconazole, aminoglutethimide, biologics (e.g., blocking antibodies to the GR), antisense ribonucleic acid (RNA), etc. In some embodiments, one or more of the therapeutic agents described above or herein are injected simultaneously or sequentially to enhance therapeutic effects (i.e., increase the adipose cells that undergo apoptosis, reduce an amount of time it takes to reduce tissue volume, and/or the like).

In addition to enabling apoptosis of non-injured adipose cells via one of more injection agents, embodiments of the present technology can include methods for planning a patient specific treatment plan, such as establishing injection parameters and/or injection sessions. Injection parameters can include the target area of tissue, the therapeutic agent injected, a depth at which the therapeutic agent is injected, a gauge of a needle used to inject the therapeutic agent, and/or the like. Injection sessions can refer to multiple rounds of injecting the therapeutic agent depending on the target area and/or desired target volume. In some embodiments, the therapeutic agent comprises multiple therapeutic agents, such as a first therapeutic agent and a second therapeutic agent. The first therapeutic agent and second therapeutic agent can be (i) different therapeutic agents injected in a singular injection session, (ii) different therapeutic agents injected in different injection sessions, or (iii) the same therapeutic agent injected in different injection sessions.

In the Figures, identical reference numbers identify generally similar, and/or identical, elements. Many of the details, dimensions, and other features shown in the Figures are merely illustrative of particular embodiments of the disclosed technology. Accordingly, other embodiments can have other details, dimensions, and features without departing from the spirit or scope of the disclosure. In addition, those of ordinary skill in the art will appreciate that further embodiments of the various disclosed technologies can be practiced without several of the details described below.

### II. Methods and Systems for Inducing Apoptosis of Adipose Cells

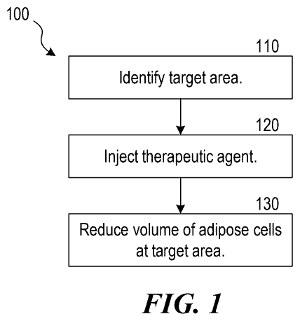

FIG. 1 is a flow diagram illustrating a method 100 for inducing apoptosis of adipose cells, in accordance with embodiments of the present technology. The method 100 includes identifying a target area (process portion 110). The target area can include a tissue area on a patient made up of adipose cells, including oropharyngeal tissue, the tongue, the soft palate, the uvula, and/or the pharyngeal wall. The target area is not limited to the oropharyngeal tissue and can also include visceral adipose tissue, white adipose tissue, and brown adipose tissue. In some embodiments, the target area includes subcutaneous fat found in the abdomen, lower flank, back, upper arms, chin, and neck. Additionally or alternatively, the target area can include tissue not exclusively in the subcutaneous fat area, such as tissue areas targeting cosmetic fat reduction and/or treatment of fat cells generally, including the lower stomach, breast, and upper and lower eyelids. The target area can also include visceral fat, for example, in the abdominal cavity. The target area can function as a guide for where the injection agent is delivered and can be a part of a patient specific treatment plan. For example, the patient specific treatment plan can include one or more target areas with the goal of targeting fat cells for OSA treatment, cosmetic fat reduction, and/or general fat cell treatment.

The method 100 further includes injecting a therapeutic agent at the target area (process portion 120). The therapeutic agent can include a ligand that is an Akt-inhibitor (e.g., ARQ092, BAY1125976, TAS-117, GSK2110183, AZD5363, GDC0068, GSK2141795, GSK690693), or a ligand that activates M2 macrophages at the target area to become proinflammatory M1 macrophages and surround non-injured adipose cells as a CLS (e.g., TDM, LPS, etc.), or an inhibitor of the GR on the ATM (e.g., mifepristone, metyrapone, ketoconazole, aminoglutethimide, biologics, and/or antisense RNA), or a ligand that induces expression of Mincle receptors to proliferate proinflammatory responses (e.g., palmitate or palmitic acid). If a therapeutic agent includes a ligand that is an Akt-inhibitor, the Akt-inhibitor can inhibit the protein kinase B (Akt) pathway and/or initiate lipolysis/cell death of the adipose cell directly. If the agent includes an inhibitor of the GR in the macrophage, the inhibitor may also cause reduction of Akt activity in adipose cells. Steroids decrease inflammation and can decrease the shift from M2 to M1 macrophages, thus decreasing CLS formation and the release of agents that interact with adipose tissue. Steroids would have a negative impact on lipolysis/adipose cell death. Blocking the GR would remove the effects of steroids and work synergistically or independently to increase adipose cell death. If the therapeutic agent includes a ligand that activates the M2 macrophages at the target area to become proinflammatory M1 macrophages and also attract M1 macrophages to the target area, the proinflammatory M1 macrophages can release pro-apoptotic factors, which can initiate cell death of the adipose cell. Without being bound by theory, all three methods for initiating cell death of adipose cells bypass having to initially injure the adipose cells, for example, by cooling the target area and/or the therapeutic agent to initiate cell death of the adipose cells.

The method 100 further includes reducing a volume of the adipose cells at the target area (process portion 130). In some embodiments, the reduction in volume is a direct result of injecting the therapeutic agent and is thus not an active step of the method 100. The volume of adipose cells reduced at the target area can correspond to a target volume based on the patient specific treatment plan which, as mentioned in process portion 110, can include a target area for fat cell treatment. Furthermore, the patient specific treatment plan can include the target volume of adipose cells at the target area needed to achieve treatment goals. For example, the patient specific treatment plan may call for a 10%, 20%, 30%, 40%, or 50% reduction of fat cells in the abdomen to achieve the patient's cosmetic goals.

The patient specific treatment plan can also include one or more of the therapeutic agents described herein. In some embodiments, the therapeutic agent injected includes an Akt-inhibitor, which can inhibit the protein kinase B (Akt) pathway and cause the adipocytes at the target area to go through apoptosis and/or cell death, therein reducing the volume of the adipose cells at the target area. In some embodiments, the injected therapeutic agent activates the M2 macrophages at the target area to become proinflammatory M1 macrophages and induce other M1 macrophages to the target area, and the proinflammatory M1 macrophages surround the adipose cell to form a CLS. M1 macrophages in the CLS can release pro-apoptotic factors, which can cause the adipocytes to go through apoptosis and/or cell death, reducing the volume of the adipose cells at the target area. In some embodiments, the injected agent is a fatty acid that induces the expression of immune receptors, which, when bound by a ligand, induce a proinflammatory response. In some embodiments, the injected agent is a GR antagonist that will act on the ATMs locally, counteracting endogenous or exogenous steroids and thus promoting M2 to M1 macrophage phenotype promoting the formation of CLS. All the agents described herein can act synergistically or independently. Thus, embodiments of the present technology can bypass having to initially injure the adipose cells (e.g., via cooling and/or cryolipolysis) to reduce the volume of adipose cells at the target area. Additionally, any or all of these agents can result in increased fibrosis in addition to reducing the fat concentration. The fibrosis can act positively for cosmetic purposes, tethering superficial layers of fat. The fibrosis can also be positive for treatment of OSA.

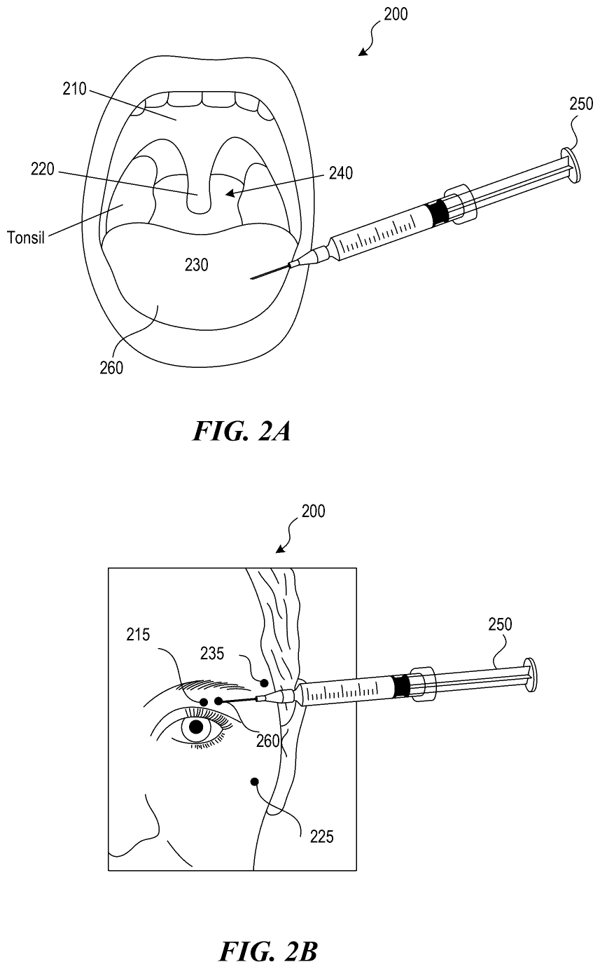

FIG. 2A illustrates a target area 260 of a human patient 200, in accordance with embodiments of the present technology. As shown in FIG. 2A, the target area 260 comprises the oropharyngeal area, but in other embodiments can include any one of the areas described with reference to FIG. 1. As shown in FIG. 2A, the oropharyngeal area can include a soft palate 210, a uvula 220, a tongue 230, and a pharyngeal wall 240. A therapeutic agent, as described in FIG. 1, is injected into the target area 260 using an injection needle 250. The injection needle 250 is used to inject the therapeutic agent. As described in FIG. 1, the patient specific treatment plan can include selecting the therapeutic agent and can use injection parameters to describe the injection of the therapeutic agent. As described herein, the injection parameters can include the therapeutic agent, a dosage for the therapeutic agent, a depth at which the therapeutic agent is injected, a gauge of a needle used to inject the therapeutic agent, a number of injection sessions, and/or the like. The injection parameters can be described in greater detail with reference to FIG. 4.

In addition to the features described in FIG. 2A, FIG. 2B illustrates the target area 260 of the human patient 200 treated for cosmetic purposes, in accordance with embodiments of the present technology. As shown in FIG. 2B, the target area 260 comprises an area near the ocular region. The ocular region can include an upper eyelid 215, a malar area 225, and a temple 235. In practice, a therapeutic agent is injected into the target area 260 at the upper eyelid 215 using the injection needle 250.

As further described herein, a patient specific treatment plan can include selecting the therapeutic agent(s) and injection parameters to describe the injection of the therapeutic agent(s) at the target area 260. In some embodiments, the injection parameters are selected in accordance with the target area's proximity to vital organs. For example, the target area 260 at the upper eyelid 215 is in close proximity to the eyes, nerves near the eyes, and blood vessels near the eyes. The injection parameters can be selected such that the injection site, needle gauge, therapeutic agent dosage, and/or the like do not negatively affect vital organs near the target area 260 at the upper eyelid 215. Current methods used to initially injure adipose cells (e.g., by cooling) make removing fat at areas near vital organs, such as at the upper and lower eyelids, especially difficult. It is worth noting that while FIGS. 2A and 2B focus on the oral cavity and ocular region, the target area 260 can include any one of the areas described herein, including subcutaneous fat and/or visceral fat for cosmetic purposes, as well as the lower stomach, abdomen, lower flank, back, upper arms, lower eyelid, chin, breast, neck, and/or the like.

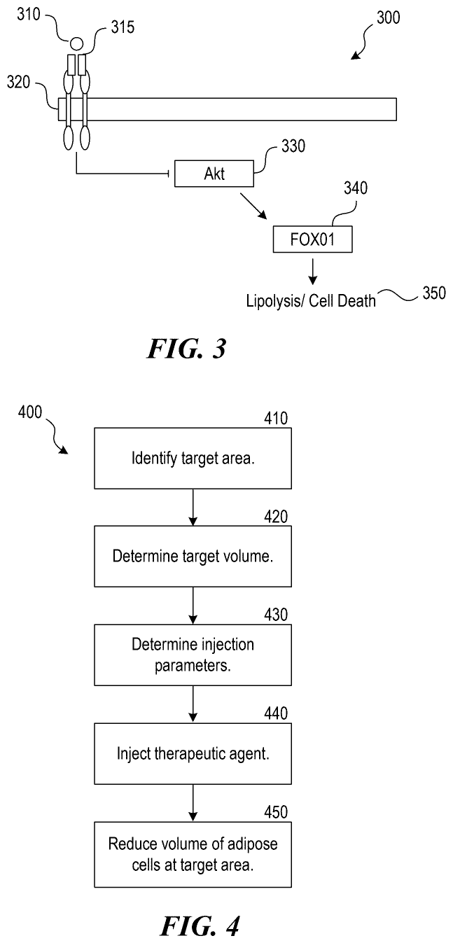

FIG. 3 illustrates a schematic cross-sectional view of an adipose cell 300 and the underlying mechanism that occurs when a therapeutic agent 310 is injected, in accordance with embodiments of the present technology. As shown in FIG. 3, the adipose cell 300 includes a cell membrane 320, and a receptor 315 for transporting the therapeutic agent 310 across the cell membrane 320. When the therapeutic agent 310 is injected at the target tissue area described elsewhere herein, the therapeutic agent 310 can bind to the receptor 315 and inhibit a protein kinase B (Akt) pathway 330 (shown schematically). The inhibition of the protein kinase B (Akt) pathway 330 causes FOX01 340 to be released, which initiates lipolysis/cell death 350 of the adipose cell.

As described herein, the therapeutic agent 310 can act as an Akt-inhibitor by binding to the receptor 315 and inhibiting the protein kinase B (Akt) pathway 330. The inhibition of the protein kinase B (Akt) pathway 330 causes the release of FOX01 340, initiating lipolysis/cell death 350 of the adipose cell. This method of initiating lipolysis/cell death of adipose cells can be done on non-injured adipose cells and evades the use of a cooling mechanism to initiate lipolysis of adipose cells.

FIG. 4 is a flow diagram illustrating a method 400 for inducing apoptosis of adipose cells, in accordance with embodiments of the present technology. The method 400 includes identifying a target area (process portion 410). The target area can include any one of the target areas described herein (e.g., with reference to FIG. 1). A physician can identify the target area using a handheld transducer, physician visualization, and/or one or more imaging modalities. For example, the imaging modalities can include ultrasound, MRI, CT scans, etc. In some embodiments, the method 400 is used to treat OSA, where the target areas are concentrated in the oropharyngeal tissue, such as the oropharynx, including the base of the tongue and the lateral pharyngeal wall. The imaging modalities and visualization techniques can navigate the oropharyngeal region and can be sensitive enough to detect adipose cell accumulation within generally smaller tissue deposits in the oral cavity to prevent excessive treatment and injury.

The method 400 further includes determining a target volume of fat cells to be reduced at the target area (process portion 420). Additionally or alternatively, the target volume can include the volume of fat cells to remain at the target area. As described in FIG. 1, the patient specific treatment plan can be used to determine the target volume of adipose cells at the target area needed to achieve treatment goals. For example, the patient specific treatment plan determines the amount of fat cells (e.g., 50%) in the target area that need to be removed.

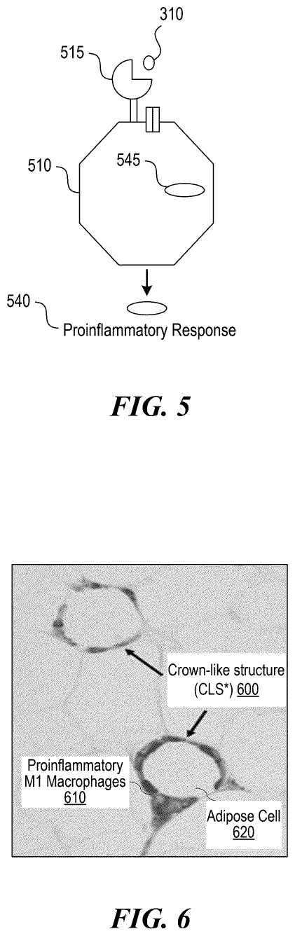

The method 400 further includes determining injection parameters (process portion 430). As described elsewhere herein, the injection parameters can include a therapeutic agent, a dosage for the therapeutic agent, a depth at which the therapeutic agent is injected, a gauge of a needle used to inject the therapeutic agent, a number of injection sessions, and/or the like. For example, the dosage of the therapeutic agent injected may vary depending on the target area and/or the target volume of reduction, as described in more detail with reference to FIG. 5. The injection parameters can be determined in accordance with the target volume determined in process portion 420.

The method 400 further includes injecting the therapeutic agent (process portion 440). The therapeutic agent can be injected at the target area determined in process portion 410 and in accordance with the injection parameters determined in process portion 430. The therapeutic agent can include an Akt-inhibitor (e.g., ARQ092, BAY1125976, TAS-117, GSK2110183, AZD5363, GDC0068, GSK2141795, GSK690693), or the therapeutic agent can activate M2 macrophages at the target area to become proinflammatory M1 macrophages (e.g., TDM, LPS, etc.). As described herein, the injected therapeutic agent can include a first therapeutic agent and a second therapeutic agent. In some embodiments, the first therapeutic agent is an Akt-inhibitor, and the second therapeutic agent activates the M2 macrophages at the target area to become proinflammatory M1 macrophages and induce CLS formation, or vice versa. Additionally or alternatively, the first therapeutic agent can be a fatty acid that induces the expression of immune receptors and the second therapeutic agent can be an Akt-inhibitor, or vice versa. In some embodiments, the first therapeutic agent is a fatty acid that induces the expression of immune receptors, and the second therapeutic agent activates M2 macrophages at the target area to become proinflammatory M1 macrophages, or vice versa. Additionally or alternatively, the first therapeutic agent can be a glucocorticoid antagonist, and the second therapeutic agent can be an Akt-inhibitor, or vice versa. In some embodiments, the first therapeutic agent is a glucocorticoid antagonist, and the second therapeutic agent activates M2 macrophages at the target area to become proinflammatory M1 macrophages, or vice versa. Additionally or alternatively, the first therapeutic agent can be a glucocorticoid antagonist, and the second therapeutic agent can be a fatty acid that induces the expression of immune receptors, or vice versa. In some embodiments, the first therapeutic agent and the second therapeutic agent are both Akt-inhibitors, or the first therapeutic agent and the second therapeutic agent both activate M2 macrophages at the target area to become proinflammatory macrophages, or the first therapeutic agent and the second therapeutic agent are both fatty acids that induce expression of immune receptors, or the first therapeutic agent and the second therapeutic agent are both glucocorticoid antagonists.

In some embodiments, the injected therapeutic agent includes a third therapeutic agent in addition to the first therapeutic agent and the second therapeutic agent. The first therapeutic agent, the second therapeutic agent, and the third therapeutic agent can be any combination of the therapeutic agents described herein. If the therapeutic agent injected is an Akt-inhibitor, the Akt-inhibitor can inhibit the protein kinase B (Akt) pathway and/or initiate lipolysis/cell death of the adipose cell directly. If the therapeutic agent injected activates the M2 macrophages at the target area to become proinflammatory M1 macrophages, the proinflammatory M1 macrophages can form CLS and release pro-apoptotic factors, which can initiate lipolysis/cell death of the adipose cell. If the therapeutic agent injected is a fatty acid that induces the expression of immune receptors, the therapeutic agent can trigger a signaling pathway (e.g., Nuclear Factor kappa-light-chain-enhancer of activated B cells (NF-κB), etc.) that increases the presence of immune receptors involved in triggering a proinflammatory response (e.g., Mincle). A ligand (e.g., a Mincle ligand from another injection) can then bind to the immunoreceptor, activating signaling pathways that lead to the production of proinflammatory cytokines, the formation of the CLS, and/or apoptosis of adipose cells. If the therapeutic agent injected is a glucocorticoid antagonist, the glucocorticoid antagonist can inhibit glucocorticoid effects in the ATM by blocking the activity and/or reducing the number of the GRs on the ATM, limiting the inhibition of endogenous or exogenous steroids to initiate lipolysis/cell death of the adipose cell. All the types of therapeutic agents injected and discussed herein initiate lipolysis/cell death of adipose cells without having to initially injure the adipose cells (e.g., using cryolipolysis).

In some embodiments, the therapeutic agent 310 includes an Akt-inhibitor and has a concentration Akt-inhibitor/Volume of solution of at least 0.1 μg/mL, 500 μg/mL, or 5 mg/mL, a range of 0.1 μg/mL to 5 mg/mL, or any value therebetween. Additionally or alternatively, the therapeutic agent 310 can include TDM and has a concentration of TDM/Volume of solution of at least 0.1 μg/mL, 500 μg/mL, or 5 mg/mL, a range of 0.1 μg/mL to 5 mg/mL, or any value therebetween. In some embodiments, the therapeutic agent 310 includes LPS and has a concentration of LPS/Volume of solution of at least at least 0.1 μg/mL, 500 μg/mL, or 5 mg/mL, a range of 0.1 μg/mL to 5 mg/mL, or any value therebetween. Additionally or alternatively, the therapeutic agent 310 can be palmitate or palmitic acid and has a concentration of palmitate or palmitic acid/Volume of solution of at least 0.1 μg/mL, 500 μg/mL, or 5 mg/mL, a range of 0.1 μg/mL to 5 mg/mL, or any value therebetween. In some embodiments, the therapeutic agent 310 includes a GR antagonist and has a concentration of GR antagonist/Volume of solution of at least 0.1 μg/mL, 500 μg/mL, or 5 mg/mL, a range of 0.1 μg/mL to 5 mg/mL, or any value therebetween. It is worth noting that the concentrations listed above may vary by plus or minus 10% therapeutic agent/Volume of solution depending on whether additional therapeutic agents are being injected concurrently or simultaneously to treat the target area. For example, it is important to control the concentration of the one or more agents injected to avoid excessive inflammation at the target area and ensure targeted adipose tissue reduction. For example, if the target area is adipose tissue at the eyelids, a generally smaller amount of therapeutic agent can be injected, such as a volume of at least 0.1 mL, 1 mL, or 5 mL, a range of 0.1 mL to 5 mL, or any value therebetween. In another example, if the target area is adipose tissue at the tongue and/or subcutaneous fat or visceral fat in the abdomen, a generally larger volume of therapeutic agent can be injected, such as a volume of at least 5 mL, 10 mL, or 100 mL, a range of 5 mL to 100 mL, or any value therebetween.

The method 400 further includes reducing a volume of adipose cells at the target area (process portion 450). The volume of adipose cells reduced at the target area can aim to achieve the target volume determined in process portion 420. For example, in some embodiments, the target volume is a 10%, 20%, 30%, 40%, or 50% reduction of fat cells (e.g., in the abdomen, lower flank, back, upper arms, chin, neck, etc.) to achieve the patient's cosmetic goals. As described herein, the therapeutic agent injected can be an Akt-inhibitor that inhibits the protein kinase B (Akt) pathway, which can directly cause the adipocytes at the target area to go through apoptosis and therein reduce the volume of the adipose cells at the target area. As also described herein, in some embodiments, the therapeutic agent injected activates the M2 macrophages at the target area to become proinflammatory M1 macrophages and causes the proinflammatory M1 macrophages to surround the adipose cell and form the CLS. macrophages in the CLS can release pro-apoptotic factors, which can cause the adipocytes to go through apoptosis and/or cell death, thereby reducing the volume of the adipose cells at the target area. Additionally or alternatively, the method 400 can include the injection of a fatty acid that induces expression of immune receptors on the macrophages at the target area. For example, the fatty acid can trigger a signaling pathway that induces expression of immune receptors associated with signaling pathways that trigger M2 macrophages at the target area to become proinflammatory macrophages. In some embodiments, the method 400 can include the injection of a glucocorticoid antagonist. The glucocorticoid antagonist can block the activity and/or reduce the number of the GRs on the ATM and can further activate M2 macrophages at the target area to become proinflammatory macrophages to surround the cell in a CLS and cause cell death of the adipocyte. The method 400 evades having to initially injure adipose cells (e.g., by cooling) to reduce the volume of adipose cells at the target area.

FIG. 5 illustrates a schematic cross-sectional view of an macrophage 510 and the underlying mechanism that occurs when a therapeutic agent is injected, in accordance with embodiments of the present technology. As shown in FIG. 5, the macrophage 510 can include a receptor 515 that the therapeutic agent 310 binds to, thereby triggering a proinflammatory response 540 of the target area macrophage 510. The M1 macrophage 510 can secrete a proinflammatory cytokine 545 to proliferate the proinflammatory response 540 and recruit other immune cells to become proinflammatory. Multiple proinflammatory M1 macrophages can form the CLS around an adipose cell in the target area (as discussed in further detail with reference to FIG. 6). The proinflammatory M1 macrophages in the CLS can further release a pro-apoptotic factor, which initiates lipolysis/cell death of the non-injured fat cell. In some embodiments, the M1 macrophage 510 is an M2 macrophage at the target area that has been activated to become a proinflammatory M1 macrophage.

In some embodiments, the M1 macrophage 510 includes a Mincle receptor as the receptor 515. The Mincle receptor can sense mycobacterium, such as tuberculosis (TB), and when activated, can induce the macrophages in the target area to shift from M2 macrophages to M1 macrophages. In some embodiments, the therapeutic agent 310 is TDM. TDM is also commonly known as a cord factor and is within the cell wall of mycobacterium (e.g., TB). TDM can bind to the receptor 515, often the Mincle receptor, and induce a proinflammatory response 540 of the M1 macrophage 510.

Additionally or alternatively, the therapeutic agent 310 can include palmitate or palmitic acid. Palmitate, a saturated fatty acid released during adipose cell lysis, can be injected into the target area to reduce adipose tissue using the body's natural immune response. Palmitate or palmitic acid can indirectly induce the expression of the receptor 515 (e.g., Mincle or various other immune receptors) by upregulating signaling pathways such as NFκKB. By inducing greater Mincle expression on Ml macrophages at the target area, a generally greater number of M1 macrophages can undergo the proinflammatory response 540. The proinflammatory response 540 of the M1 macrophages can recruit other immune cells near the target area (e.g., other M1 macrophages or M2 macrophages) to become proinflammatory, forming the CLS at the target area to promote apoptosis of adipose cells and thereby reduce adipose tissue.

In some embodiments, the therapeutic agent 310 includes a bacterial endotoxin such as LPS. LPS can be utilized in therapeutic strategies to reduce adipose tissue by promoting the shift of M2 macrophages to macrophages at the target area. When injected, LPS induces a proinflammatory response, leading to adipose-specific apoptosis. This response is characterized by the shift in macrophage polarity from M2 to M1, resulting in targeted adipose tissue reduction.

The therapeutic effects of LPS injection can be enhanced by the addition of agents such as TDM, Akt-inhibitors, palmitate, palmitic acid, and/or GR antagonists. For example, TDM or Akt-inhibitors can be co-injected with LPS to increase the shift of M2 macrophages to M1 macrophages, thereby enhancing the proinflammatory response. This enhanced activation directs M1 macrophages toward the adipose tissue to form the CLS, reducing a volume of the adipose tissue. In some embodiments, the additional agent is palmitate or palmitic acid, which enhances therapeutic effects by increasing the presence of immune receptors on the ATM at the injection site. This, in turn, improves the binding of other ligands, such as LPS, TDM, Akt-inhibitors, etc. to immune receptors, triggering signaling pathways for the proinflammatory immune response. Additionally or alternatively, the additional agent can be a GR antagonist that counteracts endogenous or exogenous steroids at the ATM, promoting the shift from M2 to M1 macrophage phenotypes and the formation of the CLS to induce adipose cell apoptosis. It is worth noting that it is important to control the concentration of LPS and the additional agents (e.g., via the injection parameters described in FIG. 4) to avoid excessive inflammation and ensure targeted adipose tissue reduction.

In some embodiments, the M1 macrophage 510 includes a GR as the receptor 515. Additionally or alternatively, the therapeutic agent 310 can be a glucocorticoid antagonist. The glucocorticoid antagonist can bind to the receptor 515, often the GR, and block the activity of the GR and/or reduce the number of the GRs on the macrophages in the target area. The binding the glucocorticoid antagonist to the GR can also further activate M2 macrophages at the target area to become proinflammatory M1 macrophages. The proinflammatory response 540 can include the secretion of the proinflammatory cytokine 545 (e.g., TNFα, IL1B, IL6, IL8, IL12, etc.) to promote the proinflammatory response of other immune cells in the target area (e.g., additional macrophages, M2 macrophages that convert to M1 macrophages, etc.). Furthermore, the binding of the therapeutic agent 310 to the receptor 515 promotes proliferation, migration, and formation of the CLS with the target area macrophage 510. M1 macrophages in the CLS can release pro-apoptotic factors, such that the pro-apoptotic factors inhibit a protein kinase B (Akt) pathway of the adipose cell and/or initiate lipolysis/cell death of the fat cells at the target area. This method of treatment bypasses having to initially injure fat cells using a cooling technique (e.g., cryolipolysis) to initiate lipolysis/cell death of fat cells at the target area.

FIG. 6 illustrates a CLS 600 around an adipose cell 620, in accordance with embodiments of the present technology. As shown in FIG. 6, the CLS 600 is made up of proinflammatory macrophages 610. The proinflammatory M1 macrophages 610 can release pro-apoptotic factors that initiate lipolysis/cell death of the adipose cell 620.

Some embodiments of the present technology activate the proinflammatory M1 macrophages 610 using the cellular processes described in FIG. 5. For example, a therapeutic agent (e.g., TDM, palmitate, LPS, GR antagonist, etc.) binds to a receptor on a target area macrophage, triggering a proinflammatory response, such as converting an M2 macrophage at the target area into a proinflammatory macrophage. The proinflammatory response can attract other immune cells to the target area. For example, the proinflammatory M1 macrophages 610 attract M2 macrophages to the target area in which the adipose cell 620 is located. Further, the proinflammatory M1 macrophages 610 can release cytokines that cause local M2 macrophages to convert to proinflammatory M1 macrophages 610. The proinflammatory M1 macrophages 610 can form the CLS 600 around the adipose cell 620 and release pro-apoptotic factors to induce apoptosis of the adipose cell 620. In some embodiments, the pro-apoptotic factors phosphorylate and inhibit a protein kinase B (Akt) pathway to initiate apoptosis of the adipose cell 620. As described in FIG. 3, the inhibition of the protein kinase B (Akt) pathway causes FOX01 to be released. The release of FOX01 can further initiate lipolysis/cell death of the adipose cell 620.

In some embodiments, the proinflammatory M1 macrophages 610 are activated by the adipose cell 620 as the adipose cell 620 is dying. When the adipose cell 620 goes through apoptosis, it can release additional factors that attract more M1 macrophages to the target area. These new M1 macrophages can undergo the cellular processes described in FIG. 5, inducing apoptosis of local adipose cells in a feed forward mechanism. The feed forward mechanism can promote adipose cell death at the target area without having to initially injure the adipose cells, for example, by cooling the target area and/or the therapeutic agent.

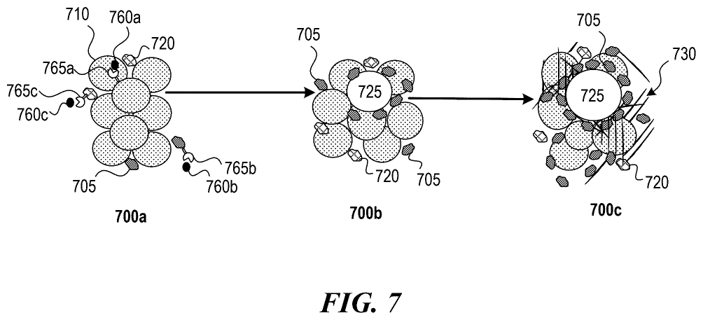

FIG. 7 illustrates a schematic cross-sectional view of groups of adipose cells 700a-c and the underlying mechanisms that occur when therapeutic agents 760a-c (collectively referred to as “therapeutic agents 760”) are injected, in accordance with embodiments of the present technology. The group of adipose cells 700a is composed of normal adipose cells 710 surrounded by M1 macrophages 705 and M2 macrophages 720. As shown in FIG. 7, one of the normal adipose cells 710 includes receptors 765a-c (collectively referred to as “receptors 765”) that the therapeutic agents 760bind to. In some embodiments, the receptors 765 and the therapeutic agents 760 are one or more of the receptors and therapeutic agents described herein. For example, the receptors 765 and the therapeutic agents 760 can include (i) a protein kinase B (Akt) pathway receptor 765a that an Akt-inhibitor 760a binds to, (ii) a Mincle receptor 765b that a Mincle receptor ligand 760b binds to, and (iii) a GR 765c that a GR antagonist 760c binds to.

The binding of the Akt-inhibitor 760a to the protein kinase B (Akt) pathway receptor 765a can inhibit a protein kinase B (Akt) pathway, thereby promoting an apoptotic and/or cell death response. As shown in the group of adipose cells 700b, the promotion of the apoptotic response can initiate the conversion of one of the normal adipose cells 710 into an apoptotic adipose cell 725 (shown schematically). The binding of the Mincle receptor ligand 760b to the Mincle receptor 765b induces a response that causes the M2 macrophages 720 to convert to M1 macrophages 705. In addition, the binding of the GR antagonist 760c to the GR 765c, as described in FIG. 1, can further induce the response, thereby increasing the M2 macrophages 720 converted to macrophages 705 and limiting glucocorticoid effects in the macrophages to promote cell death of the apoptotic adipose cell 725.

As shown in the group of adipose cells 700c, the M1 macrophages 705 can surround the non-injured adipose cell 725 to form a CLS. The M1 macrophages 705 can be proinflammatory and release pro-apoptotic factors to induce cell death of the adipose tissue and/or promote fibrosis 730. Therefore, the therapeutic agents 760 can induce the groups of adipose cells 700a-c to undergo cell death and/or fibrosis, in turn reducing a volume of adipose cells at the target area without using a cooling method to initiate injury or death of the adipose tissue and/or fibrosis.

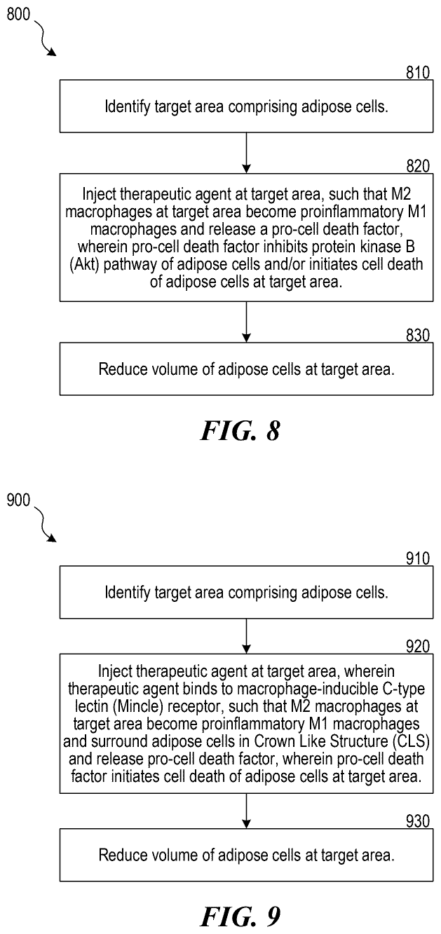

FIG. 8 is a flow diagram illustrating a method 800 for inducing apoptosis of adipose cells, in accordance with embodiments of the present technology. The method 800 can be performed in combination with one or more process portions of the methods 100 or 400 described in FIGS. 1 and 4, respectively, or any of the other methods or process portions described herein.

In some embodiments, the method 800 includes identifying a target area comprising adipose cells (process portion 810). The target area can include any one of the target areas described herein. In some embodiments, identifying the target area includes using one or more imaging modalities and/or visualization techniques to detect adipose cell accumulation within smaller tissue deposits, such as in the oral cavity, eyelids, or facial region, as well as larger tissue fat deposits, such as in the abdomen, lower flank, back, or arms, as described in more detail with reference to process portions 110 and 410 of FIGS. 1 and 4, respectively. The techniques described herein can detect variations in tissue density and/or composition, allowing for generally more accurate localization of adipose cells. For example, in the oral cavity, visualization techniques can identify small pockets of adipose tissue that may not be visible through standard examination methods. This ensures precise treatment of adipose cells and minimizes the risk of damage to surrounding non-target tissues, as described in more detail with reference to FIG. 4. In some embodiments, it is advantageous to identify the target area at the point of care using a handheld transducer or portable imaging device, allowing the physician to perform injections onsite following identification of the target area, thereby making treatment more accessible to patients outside of a surgical setting. For example, a handheld transducer can be used to determine the exact location of fat deposits around critical structures (e.g., nerves, arteries, blood vessels, eyes), which can be particularly advantageous for identifying and determining the depth of target areas where the fat deposits are generally smaller, such as the eyelids and the lateral pharyngeal wall. Similarly, the handheld transducer can identify the depth of the fat layer(s) in generally larger target areas, such as the subcutaneous layers of the abdomen and thighs, where injecting at the correct depth is also crucial to avoid complications.

The method 800 can further include injecting a therapeutic agent at the target area, such that M2 macrophages at the target area become proinflammatory M1 macrophages and release a pro-cell death factor that can inhibit a protein kinase B (Akt) pathway of the adipose cells and/or initiate the cell death of the adipose cells at the target area (process portion 820). For example, the therapeutic agent can be an Akt-inhibitor that can inhibit the protein kinase B (Akt) pathway and/or initiate cell death of the adipose cells directly, as described in more detail with reference to FIGS. 1 and 3. Additionally or alternatively, the therapeutic agent can be a Mincle receptor ligand, a fatty acid, and/or a GR antagonist, each of which can trigger various cellular signaling pathways that can enhance or initiate the proinflammatory response of macrophages and/or cell death of adipose cells at the target area, as described in more detail with reference to FIGS. 1 and 4-7. In some embodiments, adipose cell death releases factors, such as pro-apoptotic factors, that induce a feed-forward mechanism of further adipose cell death. In turn, the volume of the adipose cells at the target area can be reduced (process portion 830), as described in more detail with reference to process portions 130 and 450 of FIGS. 1 and 4, respectively.

In some embodiments, the therapeutic agent is a first therapeutic agent, and a second therapeutic agent can be injected simultaneously, consecutively, or according to a predetermined injection schedule relative to the first therapeutic agent. The second therapeutic agent can act synergistically with the first therapeutic agent to reduce the volume of adipose cells at the target area. One or more therapeutic agents (e.g., a first therapeutic agent, second therapeutic agent, third therapeutic agent, fourth therapeutic agent, etc.) can be injected at generally similar or different therapeutic concentrations, based on the target area, the target volume of cell reduction, and/or the like, as described in more detail with reference to the injection parameters of FIG. 4. The one or more therapeutic agents can include at least one of the Akt-inhibitors, Mincle receptor ligands, fatty acids, or GR antagonists described herein.

FIG. 9 is a flow diagram illustrating a method 900 for inducing apoptosis of adipose cells, in accordance with embodiments of the present technology. The method 900 can be performed in combination with one or more process portions of the method 800 described in FIG. 8 and/or any of the other methods or process portions described herein.

The method 900 can include identifying a target area (process portion 910), as described in more detail with reference to process portions 110, 410, and 810 of FIGS. 1, 4, and 8, respectively. In some embodiments, the method 900 further includes injecting a therapeutic agent at the target area that binds to a Mincle receptor, causing M2 macrophages at the target area to become proinflammatory M1 macrophages. The proinflammatory M1 macrophages can surround the adipose cells in a CLS and release a pro-cell death factor, which can initiate the cell death of the adipose cells at the target area (process portion 920). For example, the therapeutic agent can be a Mincle receptor ligand (e.g., TDM, LPS, etc.) that binds to the Mincle receptor, triggering a signaling pathway that activates M2 macrophages at the target area to become proinflammatory macrophages that surround non-injured adipose cells in a CLS, releasing pro-cell death factors, and resulting in apoptosis of the adipose cells, as described in more detail with reference to FIG. 5. Consequently, the volume of the adipose cells at the target area can be reduced (process portion 930), as described in more detail with reference to process portions 130, 450, and 830 of FIGS. 1, 4, and 8, respectively.

### III. Conclusion

It will be apparent to those having skill in the art that changes may be made to the details of the above-described embodiments without departing from the underlying principles of the present technology. In some cases, well-known structures and functions have not been shown or described in detail to avoid unnecessarily obscuring the description of the embodiments of the present technology. Although steps of methods may be presented herein in a particular order, alternative embodiments may perform the steps in a different order. Similarly, certain aspects of the present technology disclosed in the context of particular embodiments can be combined or eliminated in other embodiments. Furthermore, while advantages associated with certain embodiments of the present technology may have been disclosed in the context of those embodiments, other embodiments can also exhibit such advantages, and not all embodiments need necessarily exhibit such advantages or other advantages disclosed herein to fall within the scope of the technology. Accordingly, the disclosure and associated technology can encompass other embodiments not expressly shown or described herein.

Throughout this disclosure, the singular terms “a,” “an,” and “the” include plural referents unless the context clearly indicates otherwise. Additionally, the terms “comprising,” “including,” and “having” should be interpreted to mean including at least the recited feature(s) such that any greater number of the same feature and/or additional types of other features are not precluded.

Reference herein to “one embodiment,” “an embodiment,” “some embodiments,” or similar formulations means that a particular feature, structure, operation, or characteristic described in connection with the embodiment can be included in at least one embodiment of the present technology. Thus, the appearances of such phrases or formulations herein are not necessarily all referring to the same embodiment. Furthermore, various particular features, structures, operations, or characteristics may be combined in any suitable manner in one or more embodiments.

Unless otherwise indicated, all numbers expressing numerical values used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present technology. At the very least, and not as an attempt to limit the application of the doctrine of equivalents to the scope of the claims, each numerical parameter should at least be construed in light of the number of reported significant digits and by applying ordinary rounding techniques. Additionally, all ranges disclosed herein are to be understood to encompass any and all subranges subsumed therein. For example, a range of “1 to 10” includes any and all subranges between (and including) the minimum value of 1 and the maximum value of 10, i.e., any and all subranges having a minimum value of equal to or greater than 1 and a maximum value of equal to or less than 10, e.g., 5.5 to 10.

The disclosure set forth above is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

The present technology is illustrated, for example, according to various aspects described below as numbered clauses (1, 2, 3, etc.) for convenience. These are provided as examples and do not limit the present technology. It is noted that any of the dependent clauses may be combined in any combination, and placed into a respective independent clause. The other clauses can be presented in a similar manner.
1. A method for inducing apoptosis and/or cell death of adipose cells, the method comprising:
   identifying a target area comprising the adipose cells; and
   injecting a therapeutic agent at the target area, such that a protein kinase B (Akt) pathway of the adipose cells at the target area is inhibited and/or the cell death of the adipose cells at the target area is initiated.
2. The method of any one of the clauses herein, wherein injecting the therapeutic agent causes reduction of a volume of the adipose cells at the target area.
3. The method of any one of the clauses herein, wherein the adipose cells are non-injured adipose cells.
4. The method of any one of the clauses herein, wherein injecting the therapeutic agent comprises injecting the therapeutic agent at the target area while the therapeutic agent and the adipose cells are at room temperature or a temperature of at least 10° C.
5. The method of any one of the clauses herein, wherein the target area comprises an oropharyngeal area.
6. The method of any one of the clauses herein, wherein the target area includes one or more of a tongue, a soft palate, a uvula, or a pharyngeal wall.
7. The method of any one of the clauses herein, wherein the target area includes one or more of subcutaneous adipose tissue, visceral adipose tissue, white adipose tissue, or brown adipose tissue.
8. The method of any one of the clauses herein, wherein the target area includes an area of an abdomen, lower flank, back, upper arms, thigh, ocular region, upper eyelids, lower eyelids, chin, breast, or neck.
9. The method of any one of the clauses herein, wherein injecting the therapeutic agent initiates fibrosis of the adipose cells at the target area.
10. The method of any one of the clauses herein, wherein injecting the therapeutic agent comprises injecting the therapeutic agent to a predetermined depth based on the target area.
11. The method of any one of the clauses herein, wherein injecting the therapeutic agent comprises injecting with a needle having a gauge of 15 or less.
12. The method of any one of the clauses herein, wherein injecting the therapeutic agent comprises injecting a predetermined dosage of the therapeutic agent based on the target area and/or a desired target volume.
13. The method of any one of the clauses herein, wherein inhibiting the protein kinase B (Akt) pathway further triggers a release of FOX01, and wherein the cell death of the adipose cells is initiated by the release of FOX01.
14. The method of any one of the clauses herein, wherein the therapeutic agent includes an Akt-inhibitor, ARQ092, BAY1125976, TAS-117, GSK2110183, AZD5363, GDC0068, GSK2141795, and/or GSK690693.
15. The method of any one of the clauses herein, wherein injecting the therapeutic agent comprises injecting an Akt-inhibitor, and wherein the therapeutic agent has a concentration of at least 0.1 μg/mL of the Akt-inhibitor/Volume of solution.
16. The method of any one of the clauses herein, wherein injecting the therapeutic agent comprises injecting an Akt-inhibitor, and wherein the therapeutic agent has a concentration of at least 0.1 μg/mL, 500 μg/mL, or 5 mg/mL of the Akt-inhibitor/Volume of solution.
17. The method of any one of the clauses herein, wherein injecting the therapeutic agent comprises injecting an Akt-inhibitor, and wherein the therapeutic agent has a concentration between 0.1 μg/mL and 5 mg/mL of the Akt-inhibitor/Volume of solution.
18. A method for inducing cell death of adipose cells, the method comprising:
   identifying a target area comprising the adipose cells;
   injecting a therapeutic agent at the target area, such that M2 macrophages at the target area become proinflammatory M1 macrophages and release a pro-cell death factor, wherein the pro-cell death factor inhibits a protein kinase B (Akt) pathway of the adipose cells and/or initiates the cell death of the adipose cells at the target area; and reducing a volume of the adipose cells at the target area.
19. The method of any one of the clauses herein, wherein the cell death further attracts new macrophages to the target area, and wherein the new macrophages become proinflammatory and create a Crown-like Structure (CLS) around the adipose cells.
2. The method of any one of the clauses herein, wherein the therapeutic agent includes a macrophage-inducible C-type lectin (Mincle) receptor ligand, and wherein the Mincle receptor ligand binds to a Mincle receptor, inducing a shift in macrophage polarity at the target area from the M2 macrophages to the proinflammatory macrophages.
21. The method of claim 1, wherein the therapeutic agent binds to a macrophage-inducible C-type lectin (Mincle) receptor, such that the M2 macrophages at the target area become the proinflammatory M1 macrophages and create a Crown-like Structure (CLS) around the adipose cells, and wherein the therapeutic agent includes at least one of Trehalose-6,6′-dimycolate (TDM) or lipopolysaccharide (LPS).
22. The method of any one of the clauses herein, wherein the therapeutic agent binds to a macrophage-inducible C-type lectin (Mincle) receptor, such that the M2 macrophages at the target area become the proinflammatory M1 macrophages and create a Crown-like Structure (CLS) around the adipose cells, and wherein the therapeutic agent includes Trehalose-6,6′-dimycolate (TDM).
23. The method of any one of the clauses herein, wherein the therapeutic agent includes Trehalose-6,6′-dimycolate (TDM), and wherein the therapeutic agent has a concentration of at least 0.1 μg/mL of the TDM/Volume of solution.
24. The method of any one of the clauses herein, wherein the therapeutic agent includes Trehalose-6,6′-dimycolate (TDM), and wherein the therapeutic agent has a concentration of at least 0.1 μg/mL, 500 μg/mL, or 5 mg/mL of the TDM/Volume of solution.
25. The method of any one of the clauses herein, wherein the therapeutic agent includes Trehalose-6,6′-dimycolate (TDM), and wherein the therapeutic agent has a concentration between 0.1 μg/mL and 5 mg/mL of the TDM/Volume of solution.
26. The method of any one of the clauses herein, wherein the therapeutic agent binds to a macrophage-inducible C-type lectin (Mincle) receptor, such that the M2 macrophages at the target area become the proinflammatory M1 macrophages and create a Crown-like Structure (CLS) around the adipose cells, and wherein the therapeutic agent includes lipopolysaccharide (LPS).
27. The method of any one of the clauses herein, wherein the therapeutic agent includes lipopolysaccharide (LPS), and wherein the therapeutic agent has a concentration of at least 0.1 μg/mL of the LPS/Volume of solution.
28. The method of any one of the clauses herein, wherein the therapeutic agent includes lipopolysaccharide (LPS), and wherein the therapeutic agent has a concentration of at least 0.1 μg/mL, 500 μg/mL, or 5 mg/mL of the LPS/Volume of solution.
29. The method of any one of the clauses herein, wherein the therapeutic agent includes lipopolysaccharide (LPS), and wherein the therapeutic agent has a concentration between 0.1 μg/mL and 5 mg/mL of the LPS/Volume of solution.
30. The method of any one of the clauses herein, wherein the therapeutic agent includes a glucocorticoid receptor (GR) antagonist, such that the M2 macrophages at the target area become the proinflammatory macrophages and create a Crown-like Structure (CLS) around the adipose cells.
31. The method of any one of the clauses herein, wherein the therapeutic agent includes a glucocorticoid receptor (GR) antagonist, mifepristone, metyrapone, ketoconazole, aminoglutethimide, biologics, and/or antisense ribonucleic acid.
32. The method of any one of the clauses herein, wherein the therapeutic agent includes a glucocorticoid receptor (GR) antagonist, and wherein the therapeutic agent has a concentration of at least 0.1 μg/mL of the GR antagonist/Volume of solution.
33. The method of any one of the clauses herein, wherein the therapeutic agent includes a glucocorticoid receptor (GR) antagonist, and wherein the therapeutic agent has a concentration of at least 0.1 μg/mL, 500 μg/mL, or 5 mg/mL of the GR antagonist/Volume of solution.
34. The method of any one of the clauses herein, wherein the therapeutic agent includes a glucocorticoid receptor (GR) antagonist, and wherein the therapeutic agent has a concentration between 0.1 μg/mL and 5 mg/mL of the GR antagonist/Volume of solution.
35. The method of any one of the clauses herein, wherein the therapeutic agent includes a fatty acid that induces expression of immune receptors on macrophages at the target area, such that when a ligand binds to at least one of the immune receptors, the M2 macrophages at the target area become the proinflammatory M1 macrophages and create a Crown-like Structure (CLS) around the adipose cells.
36. The method of any one of the clauses herein, wherein the therapeutic agent comprises a fatty acid that triggers a Nuclear Factor kappa-light-chain-enhancer of activated B cells (NF-κB) signaling pathway, inducing expression of immune receptors on macrophages at the target area, such that when a ligand binds to at least one of the immune receptors, the M2 macrophages at the target area become the proinflammatory M1 macrophages and create a Crown-like Structure (CLS) around the adipose cells.
37. The method of any one of the clauses herein, wherein the therapeutic agent includes a fatty acid, palmitate, and/or palmitic acid.
38. The method of claim 1, wherein the therapeutic agent includes a fatty acid, and wherein the therapeutic agent has a concentration of at least 0.1 μg/mL of palmitate/Volume of solution and/or palmitic acid/Volume of solution.
39. The method of claim 1, wherein the therapeutic agent includes a fatty acid, and wherein the therapeutic agent has a concentration of at least 0.1 μg/mL, 500 μg/mL, or 5 mg/mL of palmitate/Volume of solution and/or palmitic acid/Volume of solution.
40. The method of claim 1, wherein the therapeutic agent includes a fatty acid, and wherein the therapeutic agent has a concentration between 0.1 μg/mL and 5 mg/ml of palmitate/Volume of solution and/or palmitic acid/Volume of solution.
41. The method of any one of the clauses herein, wherein the therapeutic agent includes palmitate and/or palmitic acid, and wherein the therapeutic agent has a concentration of at least 0.1 μg/mL of the palmitate/Volume of solution and/or the palmitic acid/Volume of solution.
42. The method of any one of the clauses herein, wherein the therapeutic agent includes palmitate and/or palmitic acid, and wherein the therapeutic agent has a concentration of at least 0.1 μg/mL, 500 μg/mL, or 5 mg/mL of the palmitate/Volume of solution and/or the palmitic acid/Volume of solution.
43. The method of any one of the clauses herein, wherein the therapeutic agent includes palmitate and/or palmitic acid, and wherein the therapeutic agent has a concentration between 0.1 μg/mL and 5 mg/mL of the palmitate/Volume of solution and/or the palmitic acid/Volume of solution.
44. The method of any one of the clauses herein, wherein injecting the therapeutic agent comprises injecting a first therapeutic agent and a second therapeutic agent based on the target area and/or desired target volume.
45. The method of any one of the clauses herein, wherein injecting the therapeutic agent comprises injecting a first therapeutic agent that includes at least one of an Akt-inhibitor, a macrophage-inducible C-type lectin (Mincle) receptor ligand, a fatty acid, or a glucocorticoid receptor (GR) antagonist and a second therapeutic agent that includes at least one of the Akt-inhibitor, the Mincle receptor ligand, the fatty acid, or the GR antagonist.
46. The method of any one of the clauses herein, wherein injecting the therapeutic agent comprises injecting a first therapeutic agent that includes palmitate or palmitic acid and a second therapeutic agent that includes lipopolysaccharide (LPS).
47. The method of any one of the clauses herein, wherein injecting the therapeutic agent comprises injecting a first therapeutic agent that includes lipopolysaccharide (LPS) and a second therapeutic agent that includes Trehalose-6,6′-dimycolate (TDM).
48. The method of any one of the clauses herein, wherein injecting the therapeutic agent comprises injecting a first therapeutic agent that includes lipopolysaccharide (LPS) and a second therapeutic agent that includes an Akt-inhibitor.
49. The method of any one of the clauses herein, wherein injecting the therapeutic agent comprises injecting a first therapeutic agent that includes lipopolysaccharide (LPS) and a second therapeutic agent that includes a glucocorticoid receptor (GR) antagonist.
50. The method of any one of the clauses herein, wherein injecting the therapeutic agent comprises injecting a first therapeutic agent, a second therapeutic agent, a third therapeutic agent, and a fourth therapeutic agent based on the target area and/or desired target volume.
51. The method of any one of the clauses herein, wherein injecting the therapeutic agent comprises injecting at least one of each of an Akt-inhibitor, a macrophage-inducible C-type lectin (Mincle) receptor ligand, a fatty acid, and a glucocorticoid receptor (GR) antagonist.
52. A method for inducing cell death of adipose cells, the method comprising:
   identifying a target area comprising the adipose cells;
   injecting a therapeutic agent at the target area, wherein the therapeutic agent binds to a macrophage-inducible C-type lectin (Mincle) receptor, such that M2 macrophages at the target area become proinflammatory macrophages and surround adipose tissue in a Crown-like Structure (CLS) and release a pro-cell death factor, wherein the pro-cell death factor initiates the cell death of the adipose cells at the target area; and reducing a volume of the adipose cells at the target area.
53. A method for inducing cell death of adipose cells, the method comprising: identifying a target area comprising the adipose cells;
   injecting a therapeutic agent at the target area, wherein the therapeutic agent inhibits a glucocorticoid receptor (GR), such that M2 macrophages at the target area become proinflammatory macrophages and form a Crown-like Structure (CLS) around adipose tissue and release a pro-cell death factor, wherein the pro-cell death factor inhibits a protein kinase B (Akt) pathway of the adipose cells and/or initiates the cell death of the adipose cells at the target area; and
   reducing a volume of the adipose cells at the target area.
54. A method for inducing cell death of adipose cells, the method comprising:
   identifying a target area comprising the adipose cells;
   injecting a therapeutic agent at the target area, wherein the therapeutic agent is a fatty acid that induces expression of immune receptors on macrophages at the target area, such that when a ligand binds to at least one of the immune receptors, M2 macrophages at the target area become proinflammatory macrophages, which surround adipose tissue in a Crown-like Structure (CLS) and release a pro-cell death factor, wherein the pro-cell death factor initiates the cell death of the adipose cells at the target area; and
   reducing a volume of the adipose cells at the target area.

I/we claim:

1. A method for inducing cell death of adipose cells, the method comprising:
identifying a target area comprising the adipose cells;
injecting a therapeutic agent at the target area, such that M2 macrophages at the target area become proinflammatory M1 macrophages and release a pro-cell death factor, wherein the pro-cell death factor inhibits a protein kinase B (Akt) pathway of the adipose cells and/or initiates the cell death of the adipose cells at the target area; and
reducing a volume of the adipose cells at the target area.

2. The method of claim 1, wherein the adipose cells are non-injured adipose cells.

3. The method of claim 1, wherein injecting the therapeutic agent comprises injecting the therapeutic agent at the target area while the therapeutic agent and the adipose cells are at room temperature or a temperature of at least 10° C.

4. The method of claim 1, wherein injecting the therapeutic agent comprises injecting the therapeutic agent to a predetermined depth based on the target area.

5. The method of claim 1, wherein injecting the therapeutic agent comprises injecting a predetermined dosage of the therapeutic agent based on the target area and/or a desired target volume.

6. The method of claim 1, wherein the target area includes one or more of subcutaneous adipose tissue, visceral adipose tissue, white adipose tissue, or brown adipose tissue.

7. The method of claim 1, wherein injecting the therapeutic agent initiates fibrosis of the adipose cells at the target area.

8. The method of claim 1, wherein inhibiting the protein kinase B (Akt) pathway further triggers a release of FOX01, and wherein the cell death of the adipose cells is initiated by the release of FOX01.

9. The method of claim 1, wherein the therapeutic agent includes an Akt-inhibitor, ARQ092, BAY1125976, TAS-117, GSK2110183, AZD5363, GDC0068, GSK2141795, and/or GSK690693.

10. The method of claim 1, wherein injecting the therapeutic agent comprises injecting an Akt-inhibitor, and wherein the therapeutic agent has a concentration of at least 0.1 μg/mL of the Akt-inhibitor/Volume of solution.

11. The method of claim 1, wherein the therapeutic agent binds to a macrophage-inducible C-type lectin (Mincle) receptor, such that the M2 macrophages at the target area become the proinflammatory M1 macrophages and create a Crown-like Structure (CLS) around the adipose cells, and wherein the therapeutic agent includes at least one of Trehalose-6,6′-dimycolate (TDM) or lipopolysaccharide (LPS).

12. The method of claim 1, wherein the therapeutic agent includes Trehalose-6,6′-dimycolate (TDM), and wherein the therapeutic agent has a concentration of at least 0.1 μg/mL of the TDM/Volume of solution.

13. The method of claim 1, wherein the therapeutic agent includes lipopolysaccharide (LPS), and wherein the therapeutic agent has a concentration of at least 0.1 μg/mL of the LPS/Volume of solution.

14. The method of claim 1, wherein the therapeutic agent includes a glucocorticoid receptor (GR) antagonist, such that the M2 macrophages at the target area become the proinflammatory M1 macrophages and create a Crown-like Structure (CLS) around the adipose cells.

15. The method of claim 1, wherein the therapeutic agent includes a glucocorticoid receptor (GR) antagonist, mifepristone, metyrapone, ketoconazole, aminoglutethimide, biologics, and/or antisense ribonucleic acid.

16. The method of claim 1, wherein the therapeutic agent includes a glucocorticoid receptor (GR) antagonist, and wherein the therapeutic agent has a concentration of at least 0.1 μg/mL of the GR antagonist/Volume of solution.

17. The method of claim 1, wherein the therapeutic agent includes a fatty acid that induces expression of immune receptors on M1 macrophages at the target area, such that when a ligand binds to at least one of the immune receptors, the M2 macrophages at the target area become the proinflammatory M1 macrophages and create a Crown-like Structure (CLS) around the adipose cells.

18. The method of claim 1, wherein the therapeutic agent includes a fatty acid, and wherein the therapeutic agent has a concentration of at least 0.1 μg/mL of palmitate/Volume of solution and/or palmitic acid/Volume of solution.

19. A method for inducing cell death of adipose cells, the method comprising:
identifying a target area comprising the adipose cells;
injecting a therapeutic agent at the target area, wherein the therapeutic agent binds to a macrophage-inducible C-type lectin (Mincle) receptor, such that M2 macrophages at the target area become proinflammatory M1 macrophages and surround the adipose cells in a Crown-like Structure (CLS) and release a pro-cell death factor, wherein the pro-cell death factor initiates the cell death of the adipose cells at the target area; and
reducing a volume of the adipose cells at the target area.

20. The method of claim 19, wherein injecting the therapeutic agent comprises injecting a first therapeutic agent that includes at least one of an Akt-inhibitor, a macrophage-inducible C-type lectin (Mincle) receptor ligand, a fatty acid, or a glucocorticoid receptor (GR) antagonist and a second therapeutic agent that includes at least one of the Akt-inhibitor, the Mincle receptor ligand, the fatty acid, or the GR antagonist.
